SEQUENCE LISTING (17 sequences)

SEQ ID NO: 1 - protein, 16 aa, Homo sapiens
MWQLLLPTALLLLVSA

SEQ ID NO: 2 - protein, 19 aa, Homo sapiens
MDWTWRILFLVAAATGAHS

SEQ ID NO: 3 - protein, 21 aa, Homo sapiens
MALPVTALLLPLALLLHAARP

SEQ ID NO: 4 - protein, 17 aa, Homo sapiens
MWLQSLLLLGTVACSIS

SEQ ID NO: 5 - protein, 107 aa, Homo sapiens
DIQMTQSTSSLSASLGDRVTISCSASQGINNYLNWYQQKPDGTVELLIYYTSTLQSGVPSRFSGSGSGTDYSLTISNLEPEDIGTYYCQQYSKLPRTFGGGTKLEIK

SEQ ID NO: 6 - protein, 122 aa, Homo sapiens
QVQLQQSGSELMMPGASVKISCKATGYTFSNYWIEWVKQRPGHGLEWIGEILPGTGRTIYNEKFKGKATFTADISSNTVQMQLSSLTSEDSAVYYCARRDYYGNFYYAMDYWGQGTSVTVSS

SEQ ID NO: 7 - protein, 17 aa, Homo sapiens
GSTSGSGKPSGEGSTKG

SEQ ID NO: 8 - protein, 5 aa, Homo sapiens
GGGGS

SEQ ID NO: 9 - protein, 15 aa, Homo sapiens
EPKSCDKTHTCPPCP

SEQ ID NO: 10 - protein, 12 aa, Homo sapiens
ESKYGPPCPSCP

SEQ ID NO: 11 - protein, 62 aa, Homo sapiens
ALSNSIMYFSHFVPVFLPAKPTTTPAPRPPTPAPTIASQPLSLRPEASRPAAGGAVHTRGLD

SEQ ID NO: 12 - protein, 80 aa, Homo sapiens
AWVSACDTEDTVGHLGPWRDKDPALWCQLCLSSQHQAIERFYDKMQNAESGRGQVMSSLAELEDDFKEGYLETVAAYYEE

SEQ ID NO: 13 - protein, 29 aa, Homo sapiens
KPFWVLVVVGGVLACYSLLVTVAFIIFWV

SEQ ID NO: 14 - protein, 41 aa, Homo sapiens
RSKRSRLLHSDYMNMTPRRPGPTRKHYQPYAPPRDFAAYRS

SEQ ID NO: 15 - protein, 24 aa, Homo sapiens
LCARPRRSPAQEDGKVYINMPGRG

SEQ ID NO: 16 - protein, 42 aa, Homo sapiens
KRGRKKLLYIFKQPFMRPVQTTQEEDGCSCRFPEEEEGGCEL

SEQ ID NO: 17 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2023/0192880 A1
(43) Pub. Date: Jun. 22, 2023

(54) CHIMERIC ANTIGEN RECEPTOR SPECIFICALLY BINDING TO CD138, IMMUNE CELL EXPRESSING SAME, AND ANTICANCER USE THEREOF

(71) Applicants: CHUNGBUK NATIONAL UNIVERSITY INDUSTRY-ACADEMIC COOPERATION FOUNDATION, Cheongju-si, Chungcheongbuk-do (KR); CELLGENTEK CO., LTD., Cheongju-si, Chungcheongbuk-do (KR)

(72) Inventors: Suk Gil SONG, Cheongju-si, Chungcheongbuk-do (KR); Hye Ran SUNG, Cheongju-si, Chungcheongbuk-do (KR)

(73) Assignees: CHUNGBUK NATIONAL UNIVERSITY INDUSTRY-ACADEMIC COOPERATION FOUNDATION, Cheongju-si, Chungcheongbuk-do (KR); CELLGENTEK CO., LTD., Cheongju-si, Chungcheongbuk-do (KR)

(21) Appl. No.: 17/765,881

(22) PCT Filed: Sep. 25, 2020
(86) PCT No.: PCT/KR2020/013118

§ 371 Date: Apr. 1, 2022

(30) Foreign Application Priority Data
Oct. 1, 2019 (KR) 10-2019-0121553

(51) Int. Cl. C07K 16/28; C12N 5/0783; C07K 14/725; C07K 14/705; C07K 14/715; C07K 14/535; C07K 14/735; A61K 35/17; A61P 35/00; A61P 7/00

(52) U.S. Cl. CPC C07K 16/2896; C12N 5/0646; C07K 14/7051; C07K 14/70521; C07K 14/70517; C07K 14/7151; C07K 14/535; C07K 14/70535; A61K 35/17; A61P 35/00; A61P 7/00; A61K 38/00

(57) ABSTRACT

Provided is a chimeric antigen receptor (CAR) specifically binding to CD138, an immune cell expressing same, and a pharmaceutical composition for the treatment or prevention of cancer including same as an active ingredient. It was confirmed that the CD138 chimeric antigen receptor (CAR)-expressing immune cell of the presently claimed subject matter efficiently exhibits strong cytotoxic ability against CD138-expressing (positive) cancer cells. Accordingly, it is expected that the CD138 chimeric antigen receptor (CAR)-expressing immune cell of the presently claimed subject matter can be utilized for the treatment of CD138-expressing (benign) cancer diseases.

### SEQUENCE LISTING

The Sequence Listing submitted in text format (.txt) filed on Sep. 14, 2022, named “SequenceListing.txt”, created on Sep. 2, 2022 (7.40 KB), is incorporated herein by reference.

### TECHNICAL FIELD

The present invention relates to a CD138 chimeric antigen receptor (CAR) specifically binding to CD138, an immune cell expressing the same, and an anticancer use thereof.

### BACKGROUND ART

CD138 or syndecan-1 (also called SYND1; SYNDECAN; SDC; SCD1; CD138 antigen, SwissProt accession number: P18827 human) is a membrane glycoprotein, which was initially described as being present on cells of epithelial origin, but was later discovered in hematopoietic cells (Sanderson, 1989). CD138 has a long extracellular domain that binds to soluble molecules (e.g., growth factors; EGF, FGF, and HGF) and insoluble molecules (e.g., extracellular matrix components, collagen, and fibronectin) via a heparan sulfate chain, and acts as a receptor for the extracellular matrix. In addition, CD138 mediates cell-cell adhesion through heparin-binding molecules expressed by adherent cells. CCD138 was shown to act as a co-receptor for growth factors in myeloma cells (Bisping, 2006). Through studies on plasma cell differentiation, it was confirmed that CD138 can be considered as a differentiation antigen (Bataille, 2006).

In malignant hematopoiesis, CD138 is highly expressed not only in MM cells, ovarian cancer, kidney tumor, gallbladder cancer, breast cancer, prostate cancer, lung cancer, colon cancer, cells of Hodgkin's and non-Hodgkin's lymphoma, chronic lymphocytic leukemia (CLL) (Horvathova, 1995), acute lymphoblastic leukemia (ALL), acute myeloblastic leukemia (AML) (Seftalioglu, 2003 (a); Seftalioglu, 2003 (b)), solid tissue sarcoma, and colon cancer, but also in most of other hematopoietic malignant tumors and solid cancers expressing CD138 (Carbone et al., 1999; Sebestyen et al., 1999; Han et al., 2004; Charnaux et al., 2004; O'Connell et al., 2004; and Orosz and Kopper, 2001).

In normal human hematopoietic constitution, CD138 expression is limited to plasma cells (Wijdenes, 1996; Chilosi, 1999), and CD138 is not expressed in peripheral blood lymphocytes, monocytes, granulocytes, and red blood cells. In particular, CD34⁺ stem cells and progenitor cells do not express CD138, and anti-CD138 monoclonal antibody (mAb) does not act on multiple colonies forming units in hematopoietic stem cell culture (Wijdenes, 1996). In the case of the non-hematopoietic system, CD138 is primarily expressed in the monolayered epithelium inside the lungs, livers, skin, kidneys, and intestines. Only weak staining was observed in endothelial cells (Bernfield, 1992; Vooijs, 1996). CD138 was reported to exist in a polymorphic form in human lymphoma cells (Gattei, 1999).

According to Surveillance, Epidemiology, and End Results (SEER) data, multiple myeloma (MM) accounts for about 1.8% of all cancers in the United States. Between 2011 and 2015, about 6.7 people per 100,000 people on average were diagnosed with multiple myeloma every year, and it was reported that about 3.3 people die each year. It is a disease that predominantly develops in the elderly people, and the average age at the time of diagnosis was 69 years, and about 30% of the cases occurred in ages between 65 and 74, and the occurrence of the disease is gradually increasing with the aging of the population. The treatment of multiple myeloma, which started with systemic chemotherapy (melphalan, etc.) in the 1960s, has been progressed to autologous hematopoietic stem cell transplantation, and target treatments (e.g., lenalidomide (an immunomodulatory agent) and bortezomib (a proteasome inhibitor), etc.) since the 2000s; and according to the development of treatments, the treatment outcomes of patients with multiple myeloma have improved significantly. Recently, monoclonal antibodies (mAb) or antibody-drug conjugates (ADC), immune-checkpoint inhibitors, anti-cancer vaccines, chimeric antigen receptor T (CAR-T) cell therapy, etc. have been studied/developed as a new treatment for multiple myeloma that can control the immune system. Since the recurrence of multiple myeloma makes the symptoms worsen and the survival rate decrease, there is an urgent need for the development of a new treatment for multiple myeloma with a new concept that can meet these medical unmet needs.

Looking at the current status of the cancer treatment market, a paradigm shift has been made from targeted therapy agent (which had been in the spotlight in the past) to immunotherapy products, and cancer treatment has advanced one step further due to the technological advancement in the immunotherapy field within 5 years, proving its effectiveness. In particular, anticancer immune cell therapy products based on T cells, which has the advantage of recognizing specific antigens of cancer cells and accurately attacking the same, have been studied most actively. CAR-T cell therapy products, which are currently under development, employ an anticancer immune mechanism and shows high efficacy and low recurrence rates, which have not been found in conventional anticancer treatment methods. However, a strong immune side effect (e.g., cytokine release syndrome (CRS), etc.), on-target toxicity derived from cancer attacking targets, especially the memory function of T cells, remain in the body for a long period of time and thereby allow the same side effects to occur at any time, and clonal expansion and memory T cells are recognized as a desirable trait to ensure efficacy and a risk factor to be controlled at the same time.

As a next-generation anti-cancer immunotherapy product that can compensate for the disadvantages of the CAR-T cell therapy product, a natural killer (NK) cell therapy product including CAR-NK cells has been drawing attention. NK cells, being one of the innate immune cells that show selective cytotoxicity against cancer cells, can recognize cancer cells immediately and remove them immediately, unlike T cells. This is because NK cells distinguish cancer cells from normal cells through various immunoreceptors on the surface of NK cells. Since NK cells can effectively remove cancer stem cells, which are most important for cancer recurrence, there is a high possibility for NK cells to prevent cancer recurrence and treat cancer effectively. Moreover, even when NK cells isolated from relatives or normal people were injected into patients in various clinical studies, there were extremely few cases where immune rejection reactions occurred unlike other immune cells, and are thus very safe even if they are developed as a cell therapy product, and their allogeneic treatment is also possible, thus having many advantages from the aspect of developing anticancer immunotherapies.

The patent documents and references mentioned in this specification are incorporated herein by reference to the same extent as if each document was individually and explicitly specified by reference.

### PRIOR ART DOCUMENT

(Patent Document 1) WO 2015/193411

(Patent Document 2) WO 2017/053889

(Patent Document 3) WO 2018/017708

(Patent Document 4) WO 2009/080829

### DISCLOSURE OF THE INVENTION

### Technical Problem

The present inventors have studied and made extensive efforts to develop an immune cell therapy product that induces cytotoxicity against CD138-expressing cancer cells. As a result, chimeric antigen receptors (CARs) having an antigen-binding domain that specifically binds to CD138 and CAR-NK cells, in which the CAR was expressed, were successfully prepared, and the potential as a cell therapy product for cancer due to the increased cytotoxic activity of CAR-NK cells were experimentally demonstrated thereby accomphlishing the present invention.

Accordingly, an object of the present invention is to provide a chimeric antigen receptor (CAR) including an antigen-binding domain that specifically binds to CD138.

Another object of the present invention is to provide a polynucleotide encoding the chimeric antigen receptor (CAR).

Still another object of the present invention is to provide a recombinant vector including the polynucleotide.

Still another object of the present invention is to provide an immune cell expressing the CD138 chimeric antigen receptor (CAR).

Still another object of the present invention is to provide a pharmaceutical composition for treating or preventing cancer, which includes immune cells expressing the CD138 chimeric antigen receptor (CAR) as an active ingredient.

Other objects and technical features of the present invention are presented in more detail by the following detailed description, claims, and drawings.

### Technical Solution

To solve the above problem,

the present invention provides a chimeric antigen receptor comprising: (i) an antigen-binding domain; (ii) a hinge region; (iii) a transmembrane domain; (iv) an intracellular co-stimulatory domain; and (v) an intracellular stimulatory signal domain, wherein the antigen-binding domain specifically binds to CD138.

Additionally, the present invention provides a polynucleotide encoding the chimeric antigen receptor.

Additionally, the present invention provides a recombinant vector including the polynucleotide.

Additionally, the present invention provides an immune cell expressing the CD138 chimeric antigen receptor (CAR).

Additionally, the present invention provides a composition for the treatment or prevention of cancer, which comprises immune cells expressing the CD138 chimeric antigen receptor (CAR) as an active ingredient.

Hereinafter, the present invention will be described in more detail.

According to one aspect of the present invention, the present invention provides a chimeric antigen receptor comprising: (i) an antigen-binding domain; (ii) a hinge region; (iii) a transmembrane domain; (iv) an intracellular co-stimulatory domain; and (v) an intracellular main stimulatory signal domain, wherein the antigen-binding domain specifically binds to CD138.

As used herein, the term “chimeric antigen receptor (CAR)” refers to a polypeptide, which includes an antigen-binding domain, a hinge region, a transmembrane domain, and an intracellular co-stimulatory domain, and an intracellular stimulatory signal domain.

While the first generation CAR includes CD3 zeta (ζ) as the intracellular signaling domain, the second generation CAR further includes at least one co-stimulatory domain derived from various proteins. The co-stimulatory domain in the second generation CAR includes, for example, CD28, CD2, 4-1BB (CD137), and OX-40 (CD134), but is not limited thereto. The third generation CAR includes two kinds of co-stimulatory domains, for example CD28, 4-1BB, OX-40, CD2, etc. but is not limited to thereto.

As used herein, the term “antigen” refers to a compound, composition, or material capable of specifically binding to a specific humoral or cellular immunity product such as an antibody molecule or T cell receptor.

As used herein, the term “antigen-binding domain” refers to any protein or polypeptide domain capable of specifically recognizing and binding an antigen target.

As used herein, the term “transmembrane domain” refers to any oligopeptide or polypeptide known to be capable of performing the function of transversing the cell membrane and linking the extracellular and intracellular stimulatory signal domains.

As used herein, the term “hinge region” refers to an amino acid stretch, which is located between the “antigen recognition and binding domain” and the “transmembrane domain” to form a flexible linker. The hinge region ensures a stable binding with an antigen by appropriately positioning the antigen-binding domain while the antigen-binding domain binds to the antigen.

As used herein, the terms intracellular “stimulatory signal domain” and “co-stimulatory domain” refer to any oligopeptide or polypeptide, which is known to function as a domain that transmit signals causing activation or inhibition of biological processes in cells.

As used herein, the intracellular domain of the CD138 chimeric antigen receptor (CAR) further comprises one or more co-stimulatory domains in addition to the stimulatory signal domain.

As used herein, the CD138 chimeric antigen receptor (CAR) comprises an antigen-binding domain that specifically binds to CD138. In the present specification, the chimeric antigen receptor of the present invention is also referred to as “CD138 chimeric antigen receptor (CAR)”.

In one embodiment of the present invention, the antigen-binding domain may be an antibody that specifically binds to CD138 or a fragment of such an antibody, and the antibody fragment may be a single chain fragment variant (scFv).

In another embodiment of the present invention, the antigen-binding domain may comprise a light chain variable region and a heavy chain variable region of an antibody.

As used herein, the term “a light chain” refers to both a full-length light chain, which includes a variable region domain V_{L} and a constant region domain CL of an antibody comprising an amino acid sequence of a variable region sufficient to impart specificity to an antigen, and a fragment thereof.

As used herein, the term “a heavy chain” refers to both a full-length heavy chain, which comprises a variable region domain V_{H} and three constant region domains CH1, CH2, and CH3 of an antibody containing an amino acid sequence of a variable region sufficient to confer specificity to an antigen, and a fragment thereof.

As used herein, the term “a light chain variable region” refers to a portion of a light chain comprising a variable region.

As used herein, the term “a heavy chain variable region” refers to a portion of a heavy chain comprising a variable region.

In still another embodiment of the present invention, the light chain variable region comprises the amino acid sequence of SEQ ID NO: 5.

In still another embodiment of the present invention, the heavy chain variable region comprises the amino acid sequence of SEQ ID NO: 6.

In still another embodiment of the present invention, a linker polypeptide positioned between the light chain variable region and the heavy chain variable region may be further included.

As used herein, the term “linker polypeptide” refers to a polypeptide that connects a light chain variable region and a heavy chain variable region to each other without impairing their original antigen-binding properties.

Accordingly, in the present invention, the linker polypeptide can be used without any limitation as long as it serves to connect the two regions to each other while maintaining the functions of the light chain variable region and the heavy chain variable region.

In a specific embodiment of the present invention, the linker polypeptide may comprise the amino acid sequence of SEQ ID NO: 7 or SEQ ID NO: 8.

In an embodiment of the present invention, the antigen-binding domain may be linked to a transmembrane domain through a hinge region.

In the present invention, the “hinge region” serves the role of a linker between an antigen-binding domain and a transmembrane domain.

In another embodiment of the present invention, the antigen-binding domain is linked to the transmembrane domain by a hinge region, and the hinge region may comprise a hinge region of IgG1, IgG4, or CD8.

In a specific embodiment of the present invention, the IgG1 hinge region comprises the amino acid sequence of SEQ ID NO: 9.

In a specific embodiment of the present invention, the IgG4 hinge region comprises the amino acid sequence of SEQ ID NO: 10.

In a specific embodiment of the present invention, the CD8 hinge region comprises the amino acid sequence of SEQ ID NO: 11.

In an embodiment of the present invention, the transmembrane domain may comprise a transmembrane domain of CD8 or CD28.

In a specific embodiment of the invention, the CD8 transmembrane domain comprises the amino acid sequence of SEQ ID NO: 12.

In a specific embodiment of the invention, the CD28 transmembrane domain compirses the amino acid sequence of SEQ ID NO: 13.

In an embodiment of the present invention, the CD138 chimeric antigen receptor may comprise an intracellular co-stimulatory domain, an intracellular stimulatory signal domain, or a combination thereof.

In the present invention, the “co-stimulatory domain” may comprise an intracellular domain of a co-stimulatory molecule selected from the group consisting of CD27, CD28, CD137 (4-1BB), DAP10, OX40, CD30, CD40, PD-1, ICOS, lymphocyte function-related antigen-1 (LFA-1), CD2, CD7, LIGHT, NKG2C, B7-H3, a ligand that specifically binds to CD83, and any combination thereof.

In a specific embodiment of the invention, the co-stimulatory domain may comprise an intracellular domain of CD28, DAP10, or CD137 (4-1BB).

In a specific embodiment of the present invention, the CD28 intracellular domain comprises the amino acid sequence of SEQ ID NO: 14.

In a specific embodiment of the present invention, the DAP10 intracellular domain comprises the amino acid sequence of SEQ ID NO: 15.

In a specific embodiment of the present invention, the CD137(4-1BB) intracellular domain comprises the amino acid sequence of SEQ ID NO: 16.

As used herein, the term “stimulatory signal domain” refers to a domain that promotes intracellular signal activation or signal amplification.

In another embodiment of the present invention, the main stimulatory signal domain may include an intracellular domain of CD3 zeta (ζ).

In a specific embodiment of the present invention, the CD3 zeta (ζ) intracellular domain comprises the amino acid sequence of SEQ ID NO: 17.

In an embodiment of the present invention, the CD138 chimeric antigen receptor may further comprise a signal peptide.

As used herein, the term “signal peptide” refers to a peptide sequence that designates the transport and location of proteins within a cell such as a specific organelle and/or a cell surface (e.g., the endoplasmic reticulum).

In a specific embodiment of the present invention, the signal peptide may be linked to an antigen-binding domain, preferably to the N-terminus of the antigen-binding domain.

In another embodiment of the present invention, the signal peptide may include a CD16, IgG, CD8, or granulocyte-macrophage colony-stimulating factor (GM-CSF) signal peptide.

In still another embodiment of the present invention, the CD16 signal peptide includes the amino acid sequence of SEQ ID NO: 1.

In still another embodiment of the present invention, the IgG signal peptide includes the amino acid sequence of SEQ ID NO: 2.

In still another embodiment of the present invention, the CD8 signal peptide includes the amino acid sequence of SEQ ID NO: 3.

In still another embodiment of the present invention, the GM-CSF signal peptide includes the amino acid sequence of SEQ ID NO: 4.

According to another aspect of the present invention, the present invention provides a polynucleotide encoding the chimeric antigen receptor.

As used herein, the term “coding” means a polynucleotide referred to as “coding for a polypeptide” when it can be transcribed and/or translated to produce mRNA for a polypeptide and/or a fragment thereof when it is manipulated by a method well known to those skilled in the art or where it is naturall occurred.

As used herein, the term “polynucleotide” is used interchangeably and refers to a polymeric form of nucleotides of any length among ribonucleotides or deoxyribonucleotides. The term polynucleotide includes a single, double, or multi-stranded DNA or RNA, genomic DNA, cDNA, DNA-RNA hybrid, or purine and pyrimidine bases or other natural, chemically or biochemically modified, unnatural, or a polymer including a derivatized nucleotide base, but is not limited thereto.

With regard to the polynucleotide encoding the chimeric antigen receptor of the present invention, it will be well understood by those skilled in the art that various modifications may be made to the coding region within a range that does not change the coding region considering the codons preferred in the organism to express the antigen receptor, and various modifications may be made within a range that does not affect the expression of the gene even in parts other than the coding region. That is, the polynucleotide of the present invention, as long as having activity equivalent thereto, may be modified by substitution, deletion, insertion, or a combination thereof, and these are also included in the scope of the present invention.

According to still another aspect of the present invention, the present invention provides a recombinant vector including the polynucleotide.

The “vector” that can be employed in the present invention may be any vectors known in the art or modified vectors in various ways. Specifically, promoter, terminator, enhancer, etc., and expression control sequences, sequences for membrane targeting or secretion, etc. in the vector can be appropriately selected and variously combined according to the purpose, depending on the type of host cell to produce the antigen receptor.

The vector of the present invention includes a plasmid vector, a cosmid vector, a bacteriophage vector, a viral vector, etc., but is not limited thereto. Suitable vectors include signal sequences or leader sequences for membrane targeting or secretion in addition to expression control elements (e.g., promoters, operators, start codons, stop codons, polyadenylation signals, and enhancers), and can be variously prepared according to the purpose.

In the present invention, the vector includes an antibiotic resistance gene commonly used as a selection marker in the art, for example, genes having resistance to ampicillin, gentamicin, carbenicillin, chloramphenicol, streptomycin, kanamycin, geneticin, neomycin, puromycin, and tetracycline.

In the present invention, the recombinant vector may be introduced into a cell.

The method of introducing the recombinant vector of the present invention into a cell may be performed using a known transfection method (e.g., a microinjection method (Capecchi, M. R., Cell 22, 479 (1980)), a calcium phosphate precipitation method (Graham, F. L. et al., Virology 52, 456 (1973)), an electroporation method (Neumann, E. et al., EMBO J. 1, 841 (1982)), a liposome-mediated transfection method (Wong, T K et al., Gene, 10, 87 (1980)), a DEAE-dextran treatment method (Gopal, Mol. Cell Biol. 5, 1188-1190 (1985)), gene bombardment (Yang et al., Proc. Natl. Acad. Sci. USA 87, 9568-9572 (1990)), etc., but are not limited thereto.

In an embodiment of the present invention, the cells into which the recombinant vector can be introduced may be immune cells, preferably NK cells, T cells, cytotoxic T cells, or regulatory T cells. Preferably, these cells may be human-derived immune cells, and more preferably human-derived NK cells.

As used herein, the term “T cell” refers to a type of lymphocyte that matures in the thymus gland. T cells play an important role in cell-mediated immunity and are distinguished from other lymphocytes (e.g., B lymphocytes) by the presence of T cell receptors on the cell surface. T cells can also be isolated or obtained from commercially available sources. T cells include all types of CD3-expressing immune cells that include helper T cells (CD4⁺ cells), cytotoxic T cells (CD8⁺ cells), natural killer T cells, regulatory T cells (Treg), and gamma-delta T cells. The “cytotoxic cells” include CD8⁺ T cells, natural-killer (NK) cells, and neutrophils that can mediate cytotoxic responses.

As used herein, the term “NK cell”, which is also known as a natural killer cell, refers to a type of lymphocyte derived from the bone marrow, which plays an important role in the innate immune system. Even in the absence of a major histocompatibility complex or antibody on the cell surface, NK cells provide a rapid immune response to virus-infected cells, tumor cells, or other stressed cells. Non-limiting examples of commercial NK cell lines include NK-92 (ATCC® CRL-2407™) and NK-92MI (ATCC® CRL-2408™) Additional examples of NK cell lines include HANK1, KHYG-1, NKL, NK-YS, NOI-90, YT, and NK101, but are not limited thereto. Non-limiting exemplary sources of such commercially available cell lines include the American Type Culture Collection or ATCC (http://www.atcc.org/) and the German Collection of Microorganisms and Cell Cultures (https://www.dsmz.de/).

In the present invention, the step of selecting the transformed cells into which the recombinant vector has been introduced can easily be performed using a phenotype expressed by the selection markers of vectors described above. For example, when the selection marker is a gene resistant to a specific antibiotic, the transformed cells can easily be selected by culturing the transformants in a medium including the antibiotic.

According to still another aspect of the present invention, there is provided a pharmaceutical composition for the treatment or prevention of cancer, comprising cells containing the recombinant vector described above as an active ingredient.

As used herein, the term “treatment” refers to (a) inhibition of the development of a disorder or disease; (b) alleviation of a disorder or disease; and (c) elimination of a disorder or disease.

As used herein, the term “prevention” refers to inhibition of the development of a disorder or disease in an animal, which has not been diagnosed as having a disorder or disease but is prone to such a disease or disease.

In an embodiment of the present invention, the cancer may be a cancer that expresses CD138.

In the present invention, non-limiting examples of the cancer may be multiple myeloma, ovarian cancer, kidney cancer, gallbladder cancer, breast cancer, prostate cancer, lung cancer, colon cancer, Hodgkin and non-Hodgkin lymphoma, chronic lymphocytic leukemia (CLL), acute lymphoblastic leukemia (ALL), acute myeloblastic leukemia (AML), solid tissue sarcoma, ovarian adenocarcinoma, bladder transitional cell carcinoma, renal clear cell carcinoma, squamous cell lung cancer, or uterine cancer.

The pharmaceutical composition of the present invention can be prepared as an injection, typically in the form of a suspension including cells. Pharmaceutical forms suitable for injection include sterile aqueous solutions or dispersions that can be ready for immediate preparation of solutions or dispersions. In all cases, pharmaceutical agents in the form of injection solutions must be sterilized and have flowability sufficient to facilitate injection.

The pharmaceutical composition of the present invention may include a pharmaceutically acceptable carrier in addition to the active ingredients.

The term “pharmaceutically acceptable” means that allergic reactions or similar adverse reactions are not caused when administered to a human. Such carriers include specific solvents, dispersion media, coating agents, antibacterial and antifungal agents, isotonic and absorption delaying agents, etc. Use of such media and agents for pharmaceutically active materials are known in the art.

The carrier of the pharmaceutical composition may be, for example, water, saline, ethanol, polyol (e.g., glycerol, propylene glycol, liquid polyethylene glycol, etc.), a suitable mixture thereof, and a solvent or dispersion medium including vegetable oil. Flowability can be maintained using a coating agent (e.g., lecithin). Various antibacterial and antifungal agents (e.g., paraben, chlorobutanol, phenol, sorbic acid, thimerosal, etc.) may be included to prevent microbial contamination, and isotonic agents (e.g., sugar, sodium chloride, etc.) may also be included. Additionally, agents that delay absorption (e.g., aluminum monostearate and gelatin) may be included in the composition so as to delay the absorption effect when administered to the body. Sterile injection solutions are prepared by mixing the required amount of the active compound in a suitable solvent including the various other ingredients mentioned above as needed, followed by sterilization by filtration.

The pharmaceutical composition of the present invention may preferably be administered by parenteral, intraperitoneal, intradermal, intramuscular, or intravenous route.

The pharmaceutical composition of the present invention is administered in a therapeutically effective amount in a manner compatible with the formulation. Additionally, the administration dose may be adjusted according to the state or condition of the subject to be treated. For parenteral administration as an aqueous injection solution, the solution must be suitably buffered as needed, and the liquid diluent is first made isotonic with sufficient saline or glucose. These special aqueous solutions are particularly suitable for intravenous, intramuscular, subcutaneous, intradermal, and intraperitoneal administration.

Information on carriers, agents, and media that can be used in the pharmaceutical composition of the present invention is known in the art (see “Remington's Pharmaceutical Sciences”, 1995, 15th edition).

### Advantageous Effects

The features and advantages of the present invention are summarized as follows:

(i) The present invention relates to a chimeric antigen receptor (CAR) specifically binding to CD138, an immune cell expressing the same, and a pharmaceutical composition for the treatment or prevention of cancer including the immune cell as an active ingredient.

(ii) It was confirmed that the CD138 chimeric antigen receptor (CAR)-expressing cells of the present invention efficiently exhibit strong cytotoxic ability against CD138-expressing (positive) cancer cells.

(iii) Therefore, it is expected that the CD138 chimeric antigen receptor (CAR)-expressing immune cells of the present invention can be utilized for the treatment or prevention of CD138-expressing (positive) cancer diseases.

### BRIEF DESCRIPTION OF THE DRAWINGS

FIG. 1 shows a graph illustrating the results of flow cytometric analysis of NK cells, in which CD138 chimeric antigen receptors (CARs) were expressed.

FIG. 2 shows a graph illustrating the cytotoxic ability of CD138 chimeric antigen receptor (CAR)-expressing NK cells against CD138-overexpressing K562 cancer cells.

FIG. 3 shows graphs illustrating the changes in the secretion of granzyme B and interferon gamma (IFN-γ) in a reaction between CD138 chimeric antigen receptor (CAR)-expressing NK cells and target cancer cells(K562) expressing CD138 (T138) or not expressing CD138 (Tneo).

FIG. 4 shows graphs illustrating the cytotoxic effect of CD138 chimeric antigen receptor (CAR)-expressing NK cells against CD138-expressing (positive) multiple myeloma (MM) cell lines RPMI8226, IM9, and MM.1R.

### MODE FOR CARRYING OUT THE INVENTION

The specific embodiments described herein represent preferred embodiments or examples of the present invention, and the scope of the present invention is not limited thereto. It will be apparent to those skilled in the art that variations and other uses of the invention do not depart from the scope of the invention described in the claims of this specification.

### EXAMPLES

Experimental Method

1. Cell Line Culture

Chronic myelogenous leukemia cancer cells K562 and three multiple myeloma (MM) cell lines of RPMI 8226, IM9, and MM1.R were cultured in an environment of 37° C. and 5% CO₂ using RPMI 1640 containing 10% FBS. The cells were used as target cells for the experiment to confirm the cytotoxic ability of NK cells. NK cells were cultured in α-MEM medium containing 12.5% FBS, 12.5% horse serum, and 0.1 mM 2-mercaptoethanol after adding 100 U/mL of recombinant IL-2, in an environment of 37° C. and 5% CO₂.

2. Design of CD138 Chimeric Antigen Receptor (CAR)

The chimeric antigen receptor (CAR) of the present invention consisted of third generation chimeric antigen receptors (CARS).

The chimeric antigen receptor (CAR) of the present invention is a third generation chimeric antigen receptor comprising the following: (i) a signal peptide; (ii) a CD138 antigen recognition and binding domain; (iii) a hinge region; (iv) a transmembrane domain; and as an intracellular stimulatory signal domain (v) a CD3 zeta (ζ) stimulatory signal domain and (vi) two co-stimulatory domains.

The amino acid sequence of each of the domains or peptides is shown in Table 1 below. The polypeptide sequence of the CD138 chimeric antigen receptor (CAR) comprising the above construction was converted into a nucleic acid sequence by codon optimization so as to be suitable for protein expression in animal cells, and the nucleic acid sequence was synthesized and cloned for use.

TABLE 1

| SEQ | | |
|---|---|---|
| ID | | |
| NO | Sequence Information | Description |
| 1 | MWQLLLPTALLLLVSA | CD16 signal |
| | | peptide |
| 2 | MDWTWRILFLVAAATGAHS | human IgG |
| | | signal |
| | | peptide |
| 3 | MALPVTALLLPLALLLHAARP | CD8 signal |
| | | peptide |
| 4 | MWLQSLLLLGTVACSIS | GM-CSF signal |
| | | peptide |
| 5 | DIQMTQSTSSLSASLGDRVTISCSASQGINNYLNWYQQKPD | variable |
| | GTVELLIYYTSTLQSGVPSRFSGSGSGTDYSLTISNLEPED | light chain |
| | IGTYYCQQYSKLPRTFGGGTKLEIK | of scFv (VL) |
| 6 | QVQLQQSGSELMMPGASVKISCKATGYTFSNYWIEWVKQR | variable |
| | PGHGLEWIGEILPGTGRTIYNEKFKGKATFTADISSNTVQ | heavy chain |
| | MQLSSLTSEDSAVYYCARRDYYGNFYYAMDYWGQGTSVTV | of scFv (VH) |
| | SS | |
| 7 | GSTSGSGKPSGEGSTKG | linker |
| | | peptide 1 |
| 8 | GGGGS | linker |
| | | peptide 2 |
| 9 | EPKSCDKTHTCPPCP | IgG1 alpha |
| | | hinge region |
| 10 | ESKYGPPCPSCP | IgG4 alpha |
| | | hinge region |
| 11 | ALSNSIMYFSHFVPVFLPAKPTTTPAPRPPTPAPTIASQP | CD8 alpha |
| | LSLRPEASRPAAGGAVHTRGLD | hinge region |
| 12 | AWVSACDTEDTVGHLGPWRDKDPALWCQLCLSSQHQAIER | CD8 alpha |
| | FYDKMQNAESGRGQVMSSLAELEDDFKEGYLETVAAYYEE | transmembrane |
| | | domain |
| 13 | KPFWVLVWGGVLACYSLLVTVAFIIFWV | CD28 |
| | | transmembrane |
| | | domain |
| 14 | RSKRSRLLHSDYMNMTPRRPGPTRKHYQPYAPPRDFAAYRS | CD28 |
| | | co-stimulatory |
| | | domain |
| 15 | LCARPRRSPAQEDGKVYINMPGRG | DAP10 |
| | | co-stimulatory |
| | | domain |
| 16 | KRGRKKLLYIFKQPFMRPVQTTQEEDGCSCRFPEEEEGGCEL | CD137(4-1BB) |
| | | co- |
| | | stimulatory |
| | | domain |
| 17 | RVKFSRSADAPAYQQGQNQLYNELNLGRREEYDVLDKRRGRD | CD3 zeta |
| | PEMGGKPRRKNPQEGLYNELQKDKMAEAYSEIGMKGERRRGK | stimulatory |
| | GHDGLYQGLSTATKDTYDALHMQALPPR | signal domain |

Specifically, each domain of the chimeric antigen receptor (CAR) in the examples of the present invention was designed as follows. That is, the chimeric antigen receptor (CAR) was prepared to include a CD16 signal peptide; a single chain variant fragment (scFv) targeting CD138 as an antigen recognition and binding domain; an extracellular domain comprising a CD8 hinge region; a transmembrane domain of CD28; an intracellular domain of CD28 and an intracellular domain of CD137 (4-1BB) as a co-stimulatory domain; and an intracellular domain of CD3 zeta (ζ) as a main stimulatory signal domain.

3. Gene Transduction

The method of introducing a cloned gene into cells was performed according to the manufacturer's manual using Lonza's Nucleofector 2B (one of the electroporation methods) and the Cell Line Nucleofector® Kit for each cell. After transformation, the cells were stabilized for 48 hours in α-MEM medium containing 12.5% FBS, 12.5% horse serum, and 0.1 mM 2-mercaptoethanol, and the transformed cells were selected by treating an antibiotic at an appropriate concentration for 2 weeks or more according to the antibiotic resistance gene of the vector used.

4. NK Cell-Mediated Cytotoxicity Assay (CFSE-7AAD Assay)

Carboxyfluorescein succinimidyl ester (CFSE) was added to target cells (T) to a final concentration of 0.5 ∞M per 1×10⁶ cells, and stained for 30 minutes in an environment of 5% CO₂ and 37° C., washed three times with PBS, 4×10⁴ cells each were dispensed into a 96-well round bottom plate. NK cells (effector cells; E) were dispensed into wells in accordance with the E:T ratio of 1:1 and various ratios thereof, and then reacted for 4 hours in an environment of 5% CO₂ and 37° C. After washing three times with PBS, the cells were suspended in 100 μL of 1% BSA/PBS, and 5 μL of 7-AAD was added to each well, reacted at 4° C. for 30 minutes with the light blocked, and washed again twice with PBS. After the NK cells were suspended in 1% BSA/PBS, the data were compared and analyzed using a flow cytometry.

5. Enzyme-Linked Immunosorbent Assay of Granzyme B and IFN-γ

After washing the NK cells once with PBS, the NK cells were added into a 96-well round bottom plate in which target cells had been seeded (4×10⁴ cells/well) with various effector target cell ratio, and then reacted in an environment of 5% CO₂ and 37° C. The level of granzyme B secreted from NK cells was measured using a human granzyme B ELISA kit, and the level of IFN-γ was measured using a human IFN-γ ELISA kit. 100 μL of the capture antibody diluted in a coating buffer was added into each well of a 96-well plate for ELISA, and the plate was sealed, coated at 4° C. overnight, washed three times with a wash buffer, and all the remaining buffer was removed. 200 μL of an assay diluent was added to each well, blocked by reacting at room temperature for one hour, washed three times with a washing buffer, and all remaining buffer was removed. Prepared 100 μL granzyme B, interferon-gamma (IFN-γ) standards or each of the samples (cell-free supernatants from each well reacted) were added thereto, and the plate was sealed, reacted at room temperature for two hours, washed five times with a wash buffer, and all the remaining buffer was removed. After adding 100 μL of a working detector (Detection Antibody+SAv-HRP reagent) to each well, the plate was sealed, reacted at room temperature for one hour, washed five times with a wash buffer, and all remaining buffer was removed. 100 μL of a substrate solution was added to each well, and after reacting at room temperature for 30 minutes with the light blocked, 100 μL of the reaction stop solution was added to each well, and the absorbance was measured at 450 nm within 20 minutes.

Experiment Results

1. Preparation and Confirming Expression of CD138 Chimeric Antigen Receptor-Expressing NK Cells (CD138CAR-NK)

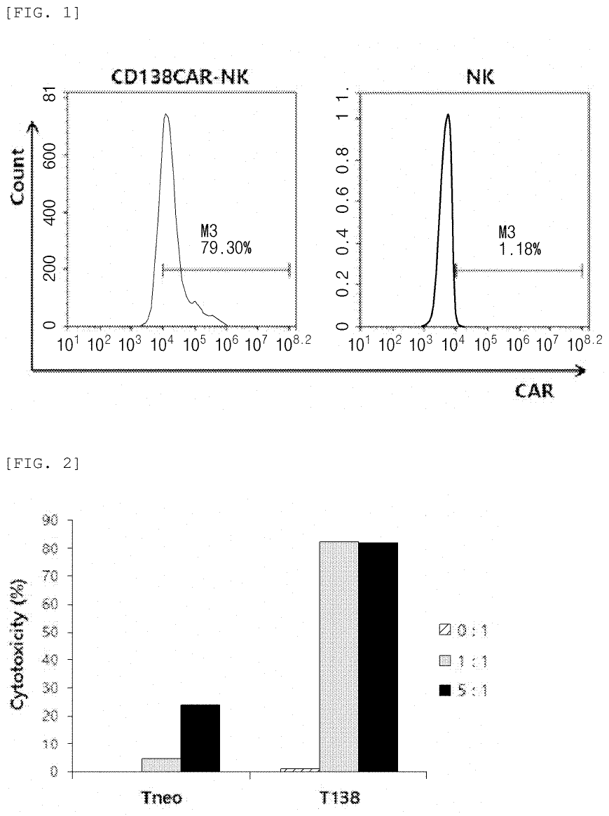

In this experiment, for more stable and efficient gene expression of NK cells, NK cells expressing CD138 chimeric antigen receptor (CAR) were produced by introducing the CD138 chimeric antigen receptor (CAR) gene by electroporation using the Lonza's Nucleofector. For the purpose of selecting only the NK cells into which the CD138 chimeric antigen receptor (CAR) gene was introduced, puromycin was used at a concentration of 1 μg/mL. As a result of confirming the expression of the CD138 chimeric antigen receptor (CAR) through flow cytometry, it was confirmed that the expression of the chimeric antigen receptor (CAR) in the finally selected NK cells was 80% or higher compared to the control NK cells (see FIG. 1).

2. Cytotoxicity of CD138 Chimeric Antigen Receptor (CAR) Expressing NK Cells (CD138CAR-NK)

In order to measure the cytotoxic ability of CD138 chimeric antigen receptor (CAR)-expressing NK cells to target cancer cells, the same was confirmed through CFSE-7AAD analysis.

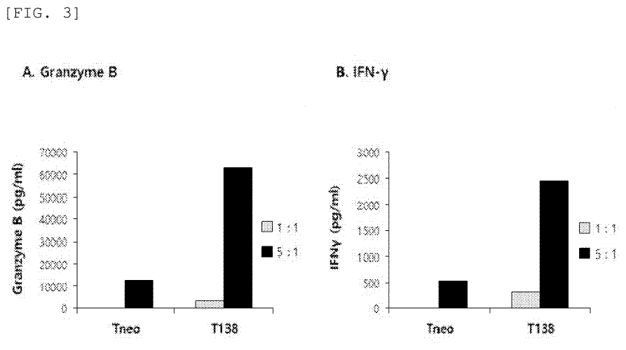

The cytotoxicity ability of CD138 chimeric antigen receptor (CAR)-expressing NK cells on K562 cells (Tneo) which do not express CD138, or K562 cells (T138) in which the CD138 antigen was artificially overexpressed, was evaluated by the E:T ratio of 0:1, 1:1, and 5:1. As a result it was confirmed that the cytotoxic effect of the CD138 chimeric antigen receptor (CAR)-expressing NK cells on K562 cells (Tneo) not expressing CD138 was about 0.3%, 5%, and 24%, whereas the cytotoxic effect on K562 cells (T138) artificially overexpressing the CD138 antigen was 0.8%, 82.6%, and 82.3%, thus showing stronger cytotoxicity. That is, it was confirmed that the CD138 chimeric antigen receptor (CAR)-expressing NK cells exhibit a cytotoxic effect specific to CD138 antigens (see FIG. 2).

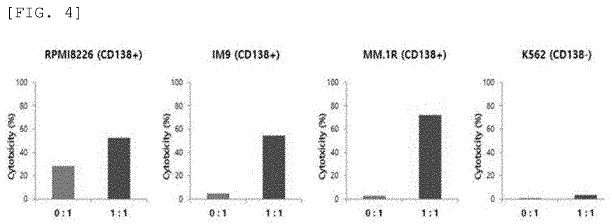

Next, the proteins such as perforin and granzyme that play an important role in destroying target cells are present in the granules of NK cells, and interferon-gamma (IFN-γ) is also an important protein whose cytotoxicity can be evaluated. As a result of measuring these proteins using ELISA, it was confirmed that granzyme B and interferon-gamma (IFN-γ) in an amount of about 5 times or more were produced specifically only in the NK cells expressing CD138 chimeric antigen receptor (CAR) that reacted with K562 (T138) expressing CD138 (see FIG. 3).

3. Anticancer Efficacy of CD138-CAR NK Cells (CD138CAR-NK) on Multiple Myeloma

The cytotoxic effect of CD138-CAR NK cells was confirmed in RPMI8226, IM9, and MM.1R, among multiple myeloma (MM) cell lines, which are CD138-expressing (positive) cells. As a control, the cytotoxic effect on CD138 non-expressing (negative) K562 cells was compared. As a result, the NK cells expressing CD138 chimeric antigen receptor (CAR) showed a high cytotoxicity of 50% or higher against all of the three types of multiple myeloma cells, and this is a result showing that the NK cells can more effectively remove CD138-expressing (positive) cancer cells by allowing the CD138 chimeric antigen receptor designed in the present invention (CAR) to effectively recognize and bind to CD138 (i.e., a target antigen) (see FIG. 4).

As described above, specific parts of the present invention have been described in detail, and it is apparent that these specific descriptions are merely preferred embodiments for those of ordinary skill in the art, and the scope of the present invention is not limited thereto. Accordingly, it should be noted that the substantial scope of the present invention is defined by the appended claims and equivalents thereof.

1. A chimeric antigen receptor comprising:
(i) an antigen-binding domain;
(ii) a hinge region;
(iii) a transmembrane domain;
(iv) an intracellular co-stimulatory domain; and
(v) an intracellular main stimulatory signal domain,
wherein the antigen-binding domain specifically binds to CD138.

2. The chimeric antigen receptor of claim 1, wherein the antigen-binding domain is an antibody or an antibody fragment, wherein the antigen-binding domain comprises a light chain variable region and a heavy chain variable region.

3. (canceled)

4. The chimeric antigen receptor of claim 2, wherein the light chain variable region comprises the amino acid sequence of SEQ ID NO: 5, and the heavy chain variable region comprises the amino acid sequence of SEQ ID NO: 6.

5. The chimeric antigen receptor of claim 2, further comprising a linker polypeptide positioned between the light chain variable region and the heavy chain variable region.

6. (canceled)

7. The chimeric antigen receptor of claim 1, wherein the antigen-binding domain is linked to the transmembrane domain by a hinge region, wherein the hinge region comprises a hinge region of IgG1, IgG4, or CD8.

8. The chimeric antigen receptor of claim 7, wherein:
the IgG1 hinge region comprises the amino acid sequence of SEQ ID NO: 9;
the IgG4 hinge region comprises the amino acid sequence of SEQ ID NO: 10; and
the CD8 hinge region comprises the amino acid sequence of SEQ ID NO: 11.

9. The chimeric antigen receptor of claim 1, wherein the transmembrane domain comprises a transmembrane domain of CD8 or CD28.

10. The chimeric antigen receptor of claim 9, wherein the CD8 transmembrane domain comprises the amino acid sequence of SEQ ID NO: 12, and the CD28 transmembrane domain comprises the amino acid sequence of SEQ ID NO: 13.

11. The chimeric antigen receptor of claim 1, wherein the intracellular co-stimulatory domain comprises an intracellular co-stimulatory domain of CD28, DAP10, or CD137 (4-1BB).

12. The chimeric antigen receptor of claim 11, wherein;
the CD28 intracellular co-stimulatory domain comprises the amino acid sequence of SEQ ID NO: 14;
the DAP10 intracellular co-stimulatory domain comprises the amino acid sequence of SEQ ID NO: 15; and
the CD137 (4-1BB) intracellular co-stimulatory domain comprises the amino acid sequence of SEQ ID NO: 16.

13. The chimeric antigen receptor of claim 1, wherein the intracellular stimulatory signal domain comprises a CD3 zeta (ζ) intracellular domain.

14. The chimeric antigen receptor of claim 13, wherein the CD3 zeta (ζ) intracellular domain comprises the amino acid sequence of SEQ ID NO: 17.

15. The chimeric antigen receptor of claim 1, further comprising a signal peptide, wherein the signal peptide comprises a signal peptide of CD16, human IgG, CD8, or GM-CSF.

16. (canceled)

17. The chimeric antigen receptor according to claim 15, wherein:
the CD16 signal peptide comprises the amino acid sequence of SEQ ID NO: 1;
the human IgG signal peptide comprises the amino acid sequence of SEQ ID NO: 2;
the CD8 signal peptide comprises the amino acid sequence of SEQ ID NO: 3; and
the GM-CSF signal peptide comprises the amino acid sequence of SEQ ID NO: 4.

18. A polynucleotide encoding the chimeric antigen receptor described in claim 1.

19. A recombinant vector comprising the polynucleotide described in claim 18.

20. A cell comprising the recombinant vector described in claim 19, wherein the cell is an NK cell, a T cell, a cytotoxic T cell, or a regulatory T cell.

21. (canceled)

22. A method for treating or preventing cancer, comprising administering a pharmaceutical composition comprising an effective amount of the cells according to claim 20 as an active ingredient to a subject in need thereof.

23. The method of claim 22, wherein the cancer is a cancer expressing CD138.

24. The method of claim 22, wherein the cancer is a cancer selected from the group consisting of multiple myeloma, ovarian cancer, kidney cancer, gallbladder cancer, breast cancer, prostate cancer, lung cancer, colon cancer, Hodgkin and non-Hodgkin lymphoma, chronic lymphocytic leukemia (CLL), acute lymphoblastic leukemia (ALL), acute myeloblastic leukemia (AML), solid tissue sarcoma, ovarian adenocarcinoma, bladder transitional cell carcinoma, renal clear cell carcinoma, squamous cell lung cancer, and uterine cancer.
